(19) United States
(12) Patent Application Publication
(10) Pub. No.: US 2018/0206735 A1
(43) Pub. Date: Jul. 26, 2018

(54) HEAD-MOUNTED DEVICE FOR CAPTURING PULSE DATA

(71) Applicant: Microsoft Technology Licensing, LLC, Redmond, WA (US)

(72) Inventors: Christian HOLZ, Seattle, WA (US); Edward Jay WANG, Hacienda Heights, CA (US)

(73) Assignee: Microsoft Technology Licensing, LLC, Redmond, WA (US)

(21) Appl. No.: 15/624,602

(22) Filed: Jun. 15, 2017

Related U.S. Application Data
(60) Provisional application No. 62/451,011, filed on Jan. 26, 2017.

(51) Int. Cl. A61B 5/0205; A61B 5/11; A61B 5/00

(52) U.S. Cl. CPC A61B 5/0205; A61B 5/11; A61B 5/02141; A61B 5/7282; A61B 5/0015; A61B 5/6803

(57) ABSTRACT

Examples are disclosed herein related to a head-mounted device, such as wearable eyeglasses, to continuously monitor pulse data and blood pressure data. One example provides a head-mounted device, comprising a first optical sensor positioned to measure pulse data at a first arterial location that is a first distance from a heart, a second optical sensor spaced apart from the first optical sensor and positioned to measure pulse data at a second arterial location that is a second, different distance from the heart, and a controller wired to the first optical sensor and the second optical sensor and configured to determine blood pressure data from the pulse data measured by the first optical sensor and the second optical sensor.

### CROSS REFERENCE TO RELATED APPLICATIONS

This application claims priority to U.S. Provisional Patent Application No. 62/451,011 filed Jan. 26, 2017, the entirety of which is hereby incorporated herein by reference.

### BACKGROUND

Blood pressure may be measured in various different manners. For example, many devices use a cuff configured to be placed around an arm to detect blood pressure via auscultatoric and/or oscillometric methods.

### SUMMARY

Examples are disclosed herein related to a head-mounted device configured to continuously monitor pulse data and blood pressure data. One example provides a head-mounted device comprising a first optical sensor positioned to measure pulse data at a first arterial location that is a first distance from a heart, a second optical sensor spaced apart from the first optical sensor and positioned to measure pulse data at a second arterial location that is a second, different distance from the heart, and a controller communicatively coupled to the first optical sensor and the second optical sensor and configured to determine blood pressure data from the pulse data measured by the first optical sensor and the second optical sensor.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 shows an example head-mounted device for monitoring pulse and blood pressure data.

FIG. 2 shows an illustration of various arteries in a human head.

FIG. 3 shows an example set of pulse wave readings from two different arteries.

FIGS. 4-7 show example implementations of head-mounted devices.

FIG. 8 is a block diagram showing an example computing system.

### DETAILED DESCRIPTION

Existing devices capable of determining blood pressure may be stationary and afford only occasional measurements (e.g., once in the morning, when going to a pharmacy once a week, or seeing a doctor once every 6 months), or may come with reduced usability, especially during everyday activities. For example, blood pressure cuffs can be used to determine absolute blood pressure, but they need to be manually attached in a resting pose for an amount of time, and thus may only be practical for occasional use.

Some wearable devices, such as fitness watches, may detect blood pressure by integrating electrocardiography (ECG) and pulse sensing. However, such sensing may involve the use of a cumbersome electrode attachment and/or active user interaction with the device to accomplish a measurement. For example, a user may need to touch a fitness watch for a period of time using the respective other hand for blood pressure measurements.

Accordingly, examples are disclosed herein related to a head-mounted device capable of continuously measuring pulse data in an unobtrusive manner from two or more optical pulse sensors positioned at different arterial locations. The optical pulse sensors may obtain two or more slightly offset pulse signals, from which pulse transit times (PTTs) and/or pulse-wave velocities may be computed to infer blood pressure changes. Based upon these pulse measurements, the head-mounted device can continuously monitor blood pressure behavior, such as postural hypotension, short-term hypertension and hypotension, without significantly impacting a user's daily routine.

Existing pulse sensing methods, such as ambulatory blood pressure (ABP) sensing, may require sensors to have contact with the body at a precise location of an artery and also may require a constant amount of pressure on the artery to detect physical expansion of the artery. In contrast, the use of optical pulse sensors as disclosed herein may detect pulse signals even if the sensors are not aligned with an artery to the extent needed for ABP sensing, may not require a constant amount of pressure on the artery, and may add little weight to a wearable device. Further, by incorporating the optical pulse sensors in a head-mounted device, the pulse sensors may naturally rest on certain arteries and provide a relatively constant pressure and contact with the skin, e.g. due to gravity. Such an implementation may avoid frequent repositioning and recalibration, which the use of a watch, wristband, chest straps, and other types of wearables may involve.

In this manner, the disclosed examples provide for frequent, unobtrusive observations of pulse and blood pressure. This may be useful to understand certain correlations between daily activities and blood pressure patterns. Further, in some examples, blood pressure data may be used in combination with motion sensor data to determine how blood pressure responds to specific actions or movements, such as occurrences of sudden hypertension and hypotension with activities such as walking, sitting down, standing up, lying down, running, climbing stairs, etc. By observing the blood pressure response to various stimuli continuously, useful insights may be determined, such as how an individual may respond to certain foods and drugs and how blood pressure varies throughout the day. Such data also may provide new understandings of existing pathologies.

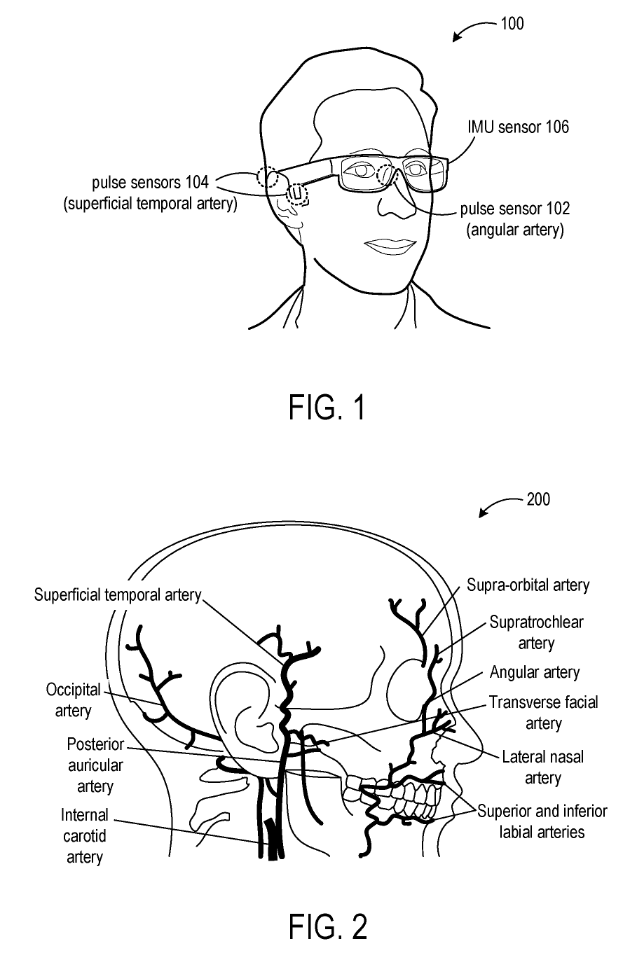

FIG. 1 shows an example head-mounted device for monitoring pulse and blood pressure in the form of a wearable device 100 similar to a pair of eyeglasses. The wearable device 100 captures the user's pulse in two or more locations of the user's head and/or face. In the depicted example, one pulse sensor 102 is positioned to measure pulse at the angular artery, while two pulse sensors 104 are positioned to measure pulse at the superficial temporal artery. More information on arterial measurement locations is described below with reference to FIG. 2. Pulse measurements may be acquired at any suitable sampling frequency. Examples include, but are not limited to, frequencies of 1 Hz to 5000 Hz.

In some examples, the wearable device 100 comprises a motion sensor, such as inertial measurement unit (IMU) sensor 106, to measure movements of the user's head and body. As mentioned above, this may allow the pulse measurements to be correlated with motion and acceleration data, and thus to help determine how blood pressure may be affected by various activities. Pulse data and blood pressure data may be observed immediately preceding or following certain detected motions, as well as over a period of time. For example, the wearable device 100 may be able to determine via motion sensors when a person stands up, and determine how quickly afterward the person's blood pressure was restored to a previous blood pressure level exhibited before standing up. Such information may provide insights related to postural hypotension. As another example, the wearable device 100 may observe how blood pressure behaves in response to eating, e.g. as inferred from detected motions or a time of day, and how long it takes before blood pressure returns to a level exhibited before eating. Thus, motion data in combination with blood pressure data may be analyzed to determine blood pressure behavior patterns associated with certain activities or events or times of day, as well as when slow or fast blood pressure recoveries tend to occur.

In some examples, motion data further may be used to determine a signal quality of the pulse measurements. For example, a certain amount or type of detected motion may indicate the potential presence of significant noise in associated pulse data, and the associated pulse data may be discarded or otherwise not utilized to determine blood pressure behavior. Thus, motion data may also be used to help obtain reliable pulse signals that are not affected by motion artifacts, providing a higher level of confidence in the blood pressure signal.

As mentioned, blood pressure can be modeled either preceding or following such motion, immediately and/or over a period of time. This may allow inquiries into questions such as how quickly did the person's blood pressure restore to its original level before the person got up (thus informing diagnoses on postural hypotension), and how blood pressure behaves after eating (e.g. as inferred from activity or time of day) along with how long it takes to recover to the normal level. More generally, this data may provide insight into patterns, both directly after an event (quick recovery vs. slow) as well as when do slow/fast recoveries occur, e.g. after what kind of activities and/or what times of day.

Any suitable motion sensing devices may be used, including but not limited to accelerometers, gyroscopes, and/or magnetometers. In some examples, the wearable device may further utilize image sensors and Global Positioning System (GPS) sensors to gather more information regarding a user's activities. In yet other examples, the glasses 100 may be in wireless communication with one or more other accompanying devices, including but not limited to other wearable computers. Other devices, such as processors or other logic devices, memory devices, batteries, communication systems, and other electronic components, also may be incorporated into the head-mounted device.

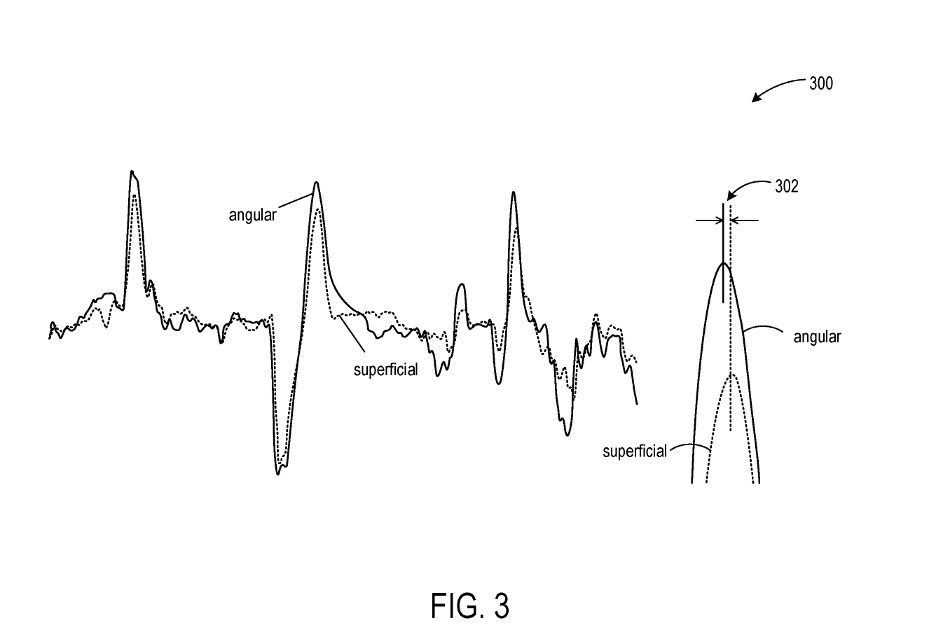

FIG. 2 shows an illustration of various arteries in a human head 200. A head-mounted device may sample pulse data from any suitable arterial locations. As an example, a device may measure pulse at the angular artery, the superficial temporal artery, and the occipital artery. Simultaneously sampling pulse at a plurality of arterial locations that are different distances from the heart allows the computation of pulse wave velocities over time, which in turn allows inference of blood pressure changes. FIG. 3 shows an example set of synchronized pulse wave readings 300 from the angular artery and the superficial temporal artery. At each pulse wave arrival, a time difference between the peaks from the two readings may be calculated as the pulse transit time, as shown at 302. Shorter pulse transit times, and thus a faster pulse wave velocity, may indicate higher blood pressure, while longer pulse transit times, and thus a slower pulse wave velocity, may indicate lower blood pressure. In some examples, ground truth data may be obtained from previously recorded blood pressure readings while a user is still to create a baseline model for use in calibrating a head-mounted device for a user.

The use of a head-mounted device, as opposed to a device worn elsewhere on the body, may provide various advantages. For example, an optical blood pressure and pulse monitoring device configured to be worn on the wrist would need a second sensor located further up the arm or require a second wearable piece, which may make such a device more cumbersome. In contrast, a head-mounted device may be configured to contact different arteries at different distances from the heart more easily, since a head-mounted device may be configured to extend at least partially around the user's head or face and thus can be made to intersect arteries at a variety of locations. A head-mounted device also does not cover parts of the body that may be crucial to everyday activities, such as hands, fingers, and feet. A head-mounted device may also be without mechanical or movable parts or sensors, which may increase its durability and robustness compared to devices with movable parts. Further, skin on the head or face is thinner compared to other locations of the body, which may provide for better optical sensing compared to other body locations. Additionally, a head-mounted device may be configured to appear as an unobtrusive, socially accepted everyday wearable device that can be inconspicuously worn in public and does not require active user input or certain user poses to monitor blood pressure behavior. For example, the eyeglass configuration of FIG. 1 allows pulse sensors to be incorporated within the natural shape of the glasses frame while touching a number of different locations for detecting useful data for blood pressure determination. Thus, whenever worn, the head-mounted device may enable continuous monitoring of blood pressure behavior without drawing unwanted attention when around other people. A head-mounted device also may avoid readjustment/retightening or precise positioning required for devices that probe specific sites on the body (e.g., watches that measure pulse may need precise contact to the skin directly above the artery). Gravity may help to apply a consistent and comfortable force on the sensor to hold the sensor in contact with the user's skin. As the gravitational force is constant, calibration, filtering, and signal design concerns may be simplified, as this force will be constant across users (though some calibration or other set-up may be used to adjust for skin color).

Further, a head-mounted device may facilitate the routing of wired connections between all sensors and an on-board controller. Such wired connections may help to ensure that readings from all sensors are properly synchronized without concerns of latency that can arise with the use of wireless connections. Wired connections to all sensors may be more easily achieved in a head-mounted device compared to devices mounted on other body parts, which can pose difficulties due to relatively large distances and/or articulating body parts being located between the sensors and controller. It will be understood that in some examples, a logic or processing system (e.g. for interpretation of pulse transit time data) may reside remotely and be wirelessly connected with the head-mounted device for data analysis and/or storage.

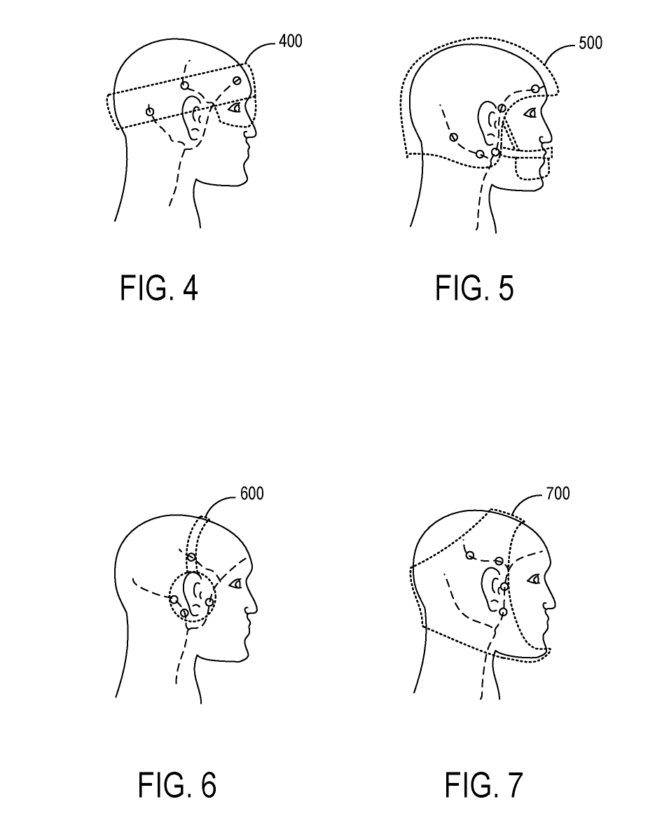

FIGS. 4-7 show other examples of head-mounted devices that include optical pulse sensors for measuring pulse data and determining blood pressure changes. Each figure shows example arterial locations (indicated as circles) on arteries (shown as dotted lines on the head) that may be sampled.

First, FIG. 4 shows an example head-mounted device in the form of a head-mounted display device 400 that may simultaneously sample pulse from a location on the occipital artery and one or more locations on the superficial temporal artery. The configuration of pulse sensors shown in FIG. 4 also may be used in a headband-shaped device.

FIG. 5 shows an example head-mounted device in the form of a helmet 500 that may measure pulse at multiple locations on each of the superficial temporal artery and the occipital artery. The helmet 500 may take any suitable form, such as a football helmet, bicycle/motorcycle helmet, or combat helmet. FIG. 6 shows an example over-ear device 600, such as earmuffs or headphones, that may likewise sample pulse at the superficial temporal and occipital arterial locations. FIG. 7 shows another example head-mounted device that takes the form of an anti-snoring mask 700. Such a mask could be used to sample pulse at the superficial temporal artery and occipital artery, as well as one or more facial arteries, during sleep. It will be understood that these head-mounted devices are shown for the purpose of example, and may sample pulse at any other suitable arterial locations than those shown. Other example head-mounted devices may include headscarves, hoods, orthopedic devices, masks, sunglasses, goggles, hats, visors, and caps.

In some embodiments, the methods and processes described herein may be tied to a computing system of one or more computing devices. In particular, such methods and processes may be implemented as a computer-application program or service, an application-programming interface (API), a library, and/or other computer-program product.

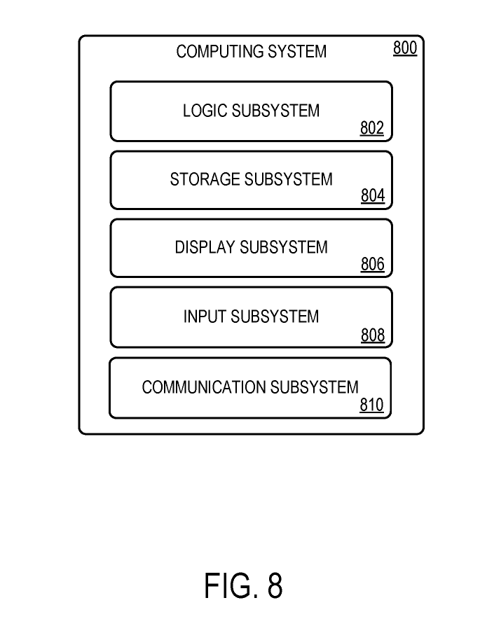

FIG. 8 schematically shows a non-limiting embodiment of a computing system 800 that can enact one or more of the methods and processes described above. Computing system 800 is shown in simplified form. Computing system 800 may take the form of one or more personal computers, server computers, tablet computers, home-entertainment computers, network computing devices, gaming devices, mobile computing devices, mobile communication devices (e.g., smart phone), and/or other computing devices. Computing system 800 may further represent the head-mounted devices 100, 400, 500, 600, and 700.

Computing system 800 includes a logic subsystem 802 and a storage subsystem 804. Computing system 800 may optionally include a display subsystem 806, input subsystem 808, communication subsystem 808, and/or other components not shown in FIG. 8.

Logic subsystem 802 includes one or more physical devices configured to execute instructions. For example, the logic subsystem 802 may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

The logic subsystem 802 may include one or more processors configured to execute software instructions. Additionally or alternatively, the logic machine may include one or more hardware or firmware logic machines configured to execute hardware or firmware instructions. Processors of the logic subsystem 802 may be single-core or multi-core, and the instructions executed thereon may be configured for sequential, parallel, and/or distributed processing. Individual components of the logic machine optionally may be distributed among two or more separate devices, which may be remotely located and/or configured for coordinated processing. Aspects of the logic subsystem 802 may be virtualized and executed by remotely accessible, networked computing devices configured in a cloud-computing configuration.

Storage subsystem 804 includes one or more physical devices configured to hold instructions executable by the logic machine to implement the methods and processes described herein. When such methods and processes are implemented, the state of storage subsystem 804 may be transformed—e.g., to hold different data.

Storage subsystem 804 may include removable and/or built-in devices. Storage subsystem 804 may include optical memory (e.g., CD, DVD, HD-DVD, Blu-Ray Disc, etc.), semiconductor memory (e.g., RAM, EPROM, EEPROM, etc.), and/or magnetic memory (e.g., hard-disk drive, floppy-disk drive, tape drive, MRAM, etc.), among others. Storage subsystem 804 may include volatile, nonvolatile, dynamic, static, read/write, read-only, random-access, sequential-access, location-addressable, file-addressable, and/or content-addressable devices.

It will be appreciated that storage subsystem 804 includes one or more physical devices. However, aspects of the instructions described herein alternatively may be propagated by a communication medium (e.g., an electromagnetic signal, an optical signal, etc.) that is not held by a physical device for a finite duration.

Aspects of logic subsystem 802 and storage subsystem 804 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

When included, display subsystem 806 may be used to present a visual representation of data held by storage subsystem 804. This visual representation may take the form of a graphical user interface (GUI). As the herein described methods and processes change the data held by the storage subsystem 804, and thus transform the state of the storage subsystem 804, the state of display subsystem 806 may likewise be transformed to visually represent changes in the underlying data. Display subsystem 806 may include one or more display devices utilizing virtually any type of technology. Such display devices may be combined with logic subsystem 802 and/or storage subsystem 804 in a shared enclosure, or such display devices may be peripheral display devices.

When included, input subsystem 808 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity.

When included, communication subsystem 810 may be configured to communicatively couple computing system 800 with one or more other computing devices. Communication subsystem 810 may include wired and/or wireless communication devices compatible with one or more different communication protocols. As non-limiting examples, the communication subsystem 810 may be configured for communication via a wireless telephone network, or a wired or wireless local- or wide-area network. In some embodiments, the communication subsystem may allow computing system 800 to send and/or receive messages to and/or from other devices via a network such as the Internet.

Another example provides a head-mounted device comprising a first optical sensor positioned to measure pulse data at a first arterial location that is a first distance from a heart, a second optical sensor spaced apart from the first optical sensor and positioned to measure pulse data at a second arterial location that is a second, different distance from the heart, and a controller wired to the first optical sensor and the second optical sensor and configured to determine blood pressure data from the pulse data measured by the first optical sensor and the second optical sensor. One or more of the first optical sensor and the second optical sensor may additionally or alternatively be positioned to measure pulse data at an angular artery. One or more of the first optical sensor and the second optical sensor may additionally or alternatively be positioned to measure pulse data at an occipital artery. One or more of the first optical sensor and the second optical sensor may additionally or alternatively be positioned to measure pulse data at a superficial temporal artery. The head-mounted device may additionally or alternatively include a third optical sensor positioned to measure pulse data at a third arterial location that is a third distance from the heart. The head-mounted device may additionally or alternatively include a motion sensor configured to monitor motion data of a wearer of the head-mounted device. The controller may additionally or alternatively be configured to determine correlation data regarding the blood pressure data and the motion data of the wearer. The first optical sensor and the second optical sensor may additionally or alternatively be configured to measure pulse arrival times at the first and second arterial locations, and the controller is configured to determine pulse transit time based on the pulse arrival times. The controller may additionally or alternatively be configured to determine changes in blood pressure based upon changes in the pulse transit time. The head-mounted device may additionally or alternatively include an eyeglass device.

Another example provides a head-mounted device comprising a first optical sensor positioned to measure pulse data at a first arterial location that is a first distance from a heart, a second optical sensor spaced apart from the first optical sensor and positioned to measure pulse data at a second arterial location that is a second, different distance from the heart, a motion sensor configured to monitor motion data of a wearer, and a controller wired to the first optical sensor and the second optical sensor and configured to determine blood pressure data from the pulse data measured by the first optical sensor and the second optical sensor, and to determine correlation data regarding the blood pressure data and the motion data of the wearer. One or more of the first optical sensor and the second optical sensor may additionally or alternatively be positioned to measure pulse data at an angular artery. One or more of the first optical sensor and the second optical sensor may additionally or alternatively be positioned to measure pulse data at an occipital artery. One or more of the first optical sensor and the second optical sensor may additionally or alternatively be positioned to measure pulse data at a superficial temporal artery. The first optical sensor and the second optical sensor may additionally or alternatively be configured to measure pulse arrival times at the first and second arterial locations, and the controller may additionally or alternatively be configured to determine pulse transit time based on the pulse arrival times. The controller may additionally or alternatively be configured to determine changes in blood pressure based upon changes in the pulse transit time. The head-mounted device may additionally or alternatively include an eyeglass device.

Another example includes a wearable eyeglass device comprising a first optical sensor positioned to measure pulse data at an angular arterial location on a head, a second optical sensor spaced apart from the first optical sensor and positioned to measure pulse data at a superficial temporal arterial location on the head, and a controller wired to the first optical sensor and the second optical sensor and configured to determine blood pressure data from the pulse data measured by the first optical sensor and the second optical sensor. The head-mounted device may additionally or alternatively include a motion sensor configured to monitor motion data of a wearer of the head-mounted device. The controller may additionally or alternatively be configured to determine correlation data regarding the blood pressure data and the motion data of the wearer.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A head-mounted device, comprising:
a first optical sensor positioned to measure pulse data at a first arterial location that is a first distance from a heart;
a second optical sensor spaced apart from the first optical sensor and positioned to measure pulse data at a second arterial location that is a second, different distance from the heart; and
a controller wired to the first optical sensor and the second optical sensor and configured to determine blood pressure data from the pulse data measured by the first optical sensor and the second optical sensor.

2. The head-mounted device of claim 1, wherein one or more of the first optical sensor and the second optical sensor is positioned to measure pulse data at an angular artery.

3. The head-mounted device of claim 1, wherein one or more of the first optical sensor and the second optical sensor is positioned to measure pulse data at an occipital artery.

4. The head-mounted device of claim 1, wherein one or more of the first optical sensor and the second optical sensor is positioned to measure pulse data at a superficial temporal artery.

5. The head-mounted device of claim 1, further comprising a third optical sensor positioned to measure pulse data at a third arterial location that is a third distance from the heart.

6. The head-mounted device of claim 1, further comprising a motion sensor configured to monitor motion data of a wearer of the head-mounted device.

7. The head-mounted device of claim 6, wherein the controller is configured to determine correlation data regarding the blood pressure data and the motion data of the wearer.

8. The head-mounted device of claim 1, wherein the first optical sensor and the second optical sensor are configured to measure pulse arrival times at the first and second arterial locations, and the controller is configured to determine pulse transit time based on the pulse arrival times.

9. The head-mounted device of claim 8, wherein the controller is configured to determine changes in blood pressure based upon changes in the pulse transit time.

10. The head-mounted device of claim 1, wherein the head-mounted device comprises an eyeglass device.

11. A head-mounted device, comprising:
a first optical sensor positioned to measure pulse data at a first arterial location that is a first distance from a heart;
a second optical sensor spaced apart from the first optical sensor and positioned to measure pulse data at a second arterial location that is a second, different distance from the heart;
a motion sensor configured to monitor motion data of a wearer; and
a controller wired to the first optical sensor and the second optical sensor and configured to determine blood pressure data from the pulse data measured by the first optical sensor and the second optical sensor, and to determine correlation data regarding the blood pressure data and the motion data of the wearer.

12. The head-mounted device of claim 11, wherein one or more of the first optical sensor and the second optical sensor is positioned to measure pulse data at an angular artery.

13. The head-mounted device of claim 11, wherein one or more of the first optical sensor and the second optical sensor is positioned to measure pulse data at an occipital artery.

14. The head-mounted device of claim 11, wherein one or more of the first optical sensor and the second optical sensor is positioned to measure pulse data at a superficial temporal artery.

15. The head-mounted device of claim 11, wherein the first optical sensor and the second optical sensor are configured to measure pulse arrival times at the first and second arterial locations, and the controller is configured to determine pulse transit time based on the pulse arrival times.

16. The head-mounted device of claim 15, wherein the controller is configured to determine changes in blood pressure based upon changes in the pulse transit time.

17. The head-mounted device of claim 11, wherein the head-mounted device comprises an eyeglass device.

18. A wearable eyeglass device, comprising:
a first optical sensor positioned to measure pulse data at an angular arterial location on a head;
a second optical sensor spaced apart from the first optical sensor and positioned to measure pulse data at a superficial temporal arterial location on the head; and
a controller wired to the first optical sensor and the second optical sensor and configured to determine blood pressure data from the pulse data measured by the first optical sensor and the second optical sensor.

19. The wearable eyeglass device of claim 18, further comprising a motion sensor configured to monitor motion data of a wearer of the head-mounted device.

20. The wearable eyeglass device of claim 19, wherein the controller is configured to determine correlation data regarding the blood pressure data and the motion data of the wearer.
